(19) United States
(12) Patent Application Publication
(10) Pub. No.: US 2018/0064817 A1
(43) Pub. Date: Mar. 8, 2018

(54) Tamper Resistant Pharmaceutical Composition

(71) Applicants: Jayendrakumar Dasharathlal PATEL, Vijapur (IN); Shwetaben Dasharathbhai Patel, Vijapur (IN)

(72) Inventors: Jayendrakumar Dasharathlal Patel, Vijapur (IN); Kaushikakumari Jayendrakumar Patel, Vijapur (IN); Shwetaben Dasharathbhai Patel, Vijapur (IN)

(73) Assignee: Jayendrakumar Dasharathlal Patel, Vijapur (IN)

(21) Appl. No.: 15/549,167

(22) PCT Filed: Dec. 29, 2016
(86) PCT No.: PCT/IB2016/058081

§ 371 Date: Aug. 6, 2017

(30) Foreign Application Priority Data
Apr. 23, 2016 (IN) 201621014201

(51) Int. Cl. A61K 47/38; A61K 9/20; A61K 47/32; A61K 47/34; A61K 47/12; A61K 47/02

(52) U.S. Cl. CPC A61K 47/38; A61K 9/2054; A61K 47/02; A61K 47/34; A61K 47/12; A61K 47/32

(57) ABSTRACT

Tamper resistant pharmaceutical composition and a process for the production of tamper resistant pharmaceutical composition are described herein. Tamper resistant pharmaceutical composition comprising drug substance and water soluble ingredient having cloud point greater than about 90° C., wherein the cloud point of the water soluble ingredient inhibit extraction of the drug substance from the pharmaceutical composition by hot water having temperature greater than about 90° C. Tamper resistant pharmaceutical composition comprising (a) drug substance; (b) water soluble ingredient; and (c) cloud point modifier, wherein the cloud point modifier increase the cloud point of the water soluble ingredient till at least about 90° C.; wherein the water soluble ingredient and the cloud point modifier inhibit extraction of the drug substance from the pharmaceutical composition by hot water having temperature greater than about 90° C. Tamper resistant pharmaceutical composition is prepared by thermal or non-thermal process.

### FIELD OF THE INVENTION

The present invention is relates to tamper resistant pharmaceutical composition and a process for the production of tamper resistant pharmaceutical composition.

### BACKGROUND OF THE INVENTION

When drugs are used in a manner or amount inconsistent with the medical or social patterns of a culture, it is called drug abuse.

The currently available dosage forms comprising pharmaceutically active substance which has high potential for drug abuse are subject to misuse by tampering with dosage form and administering the drug by any route other than instructed such as parenteral route or nasal route.

With oral solid dosage forms containing these active agents, drug abusers may crush oral solid dosage forms to powders and/or extract the active agents from the dosage forms and then self-administer the extracted active agents by injection, intranasally or by inhalation (e.g., by snorting). One common method of extracting drug from its dosage form is by first mixing intact or crushed dosage form with a suitable liquid (e.g., water) and then filtering and/or extracting the drug from the mixture for intravenous injection. Typically, abusers dissolve the crushed dosage form in from about 0.1 ml to about 5 ml of a solvent (e.g., water) and then inject the resulting solution, suspension or gel intravenously.

Because the potential for abuse of prescription drug products has become an important issue for the world, the pharmaceutical industry is striving to develop abuse resistant formulations in order to reduce the potential for misuse of prescription drugs. Various concepts for the avoidance of drug abuse have been developed by many pharmaceutical companies by adding aversion agent in dosage form, by making pill harder to resist crushing, by adding gelling agent to resist parenteral abuse, by making multiparticulates dosage form comprising lipidic and wax materials to resist extraction, etc.

Hence, the present invention is aimed at the deterrence of abuse and illegal attempts to extract the drug substance from pharmaceutical drug products, especially those active agents that are water soluble, by hot water having temperature greater than about 90° C. and thereby creating difficulties in getting pure drug substance for abuse, particularly via parenteral and nasal route. To the best of our knowledge, existing prior art does not teach about extraction of the drug substance from the dosage form by hot water having temperature greater than about 90° C. and which crucial property of water soluble ingredient must required to inhibit extraction of the drug substance form the pharmaceutical composition by hot water having temperature greater than about 90° C.

### OBJECTS OF THE INVENTION

It is an object of certain embodiments of the present invention to provide tamper resistant pharmaceutical composition comprising at least one drug substance and at least one water soluble ingredient, wherein cloud point of the water soluble ingredient is greater than about 90° C.

It is an object of certain embodiments of the present invention to provide tamper resistant pharmaceutical composition comprising (a) at least one drug substance; (b) at least one water soluble ingredient having cloud point lower than about 90° C.; and (c) at least one cloud point modifier, wherein the cloud point modifier increase the cloud point of the water soluble ingredient till at least about 90° C.

It is an object of certain embodiments of the present invention to provide tamper resistant pharmaceutical composition which is adapted to reduce or mitigate potential abuse by making it more difficult for the pharmaceutical active ingredient to be extracted using hot water having temperature greater than about 90° C. and used or administered in a manner other than that originally intended.

It is an object of certain embodiments of the present invention to provide tamper resistant pharmaceutical composition which is resistant to tampering (e.g., crushing, grinding, chewing or a combination thereof).

It is an object of certain embodiments of the present invention to provide tamper resistant pharmaceutical composition comprising an active agent susceptible to abuse, which is subject to less parenteral abuse than other dosage forms.

It is an object of certain embodiments of the present invention to provide tamper resistant pharmaceutical composition comprising an active agent susceptible to abuse, which is subject to less intranasal abuse than other dosage forms.

It is an object of certain embodiments of the present invention to provide tamper resistant pharmaceutical composition comprising an active agent susceptible to abuse, which is subject to less oral abuse than other dosage forms.

It is a further object of certain embodiments of the present invention to treat a disease or condition by administering tamper resistant pharmaceutical composition as disclosed herein to a patient in need thereof.

It is a further object of certain embodiments of the present invention to provide a manufacturing method of tamper resistant pharmaceutical composition; wherein the pharmaceutical composition is manufactured by thermal or non-thermal process, wherein the composition prepare by thermal process exhibit higher breaking strength compared to prepare by non-thermal process.

### SUMMARY OF THE INVENTION

Majority of drug substances having abuse potentiality are water soluble. Tamper resistant pharmaceutical composition comprising drug substance along with water soluble ingredient add additional extraction step in separation of drug substance from water soluble ingredients or from dosage form. So it creates complexity in getting pure drug substance from dosage form without extraction step. However, it is surprisingly found that, all water soluble ingredients are not suitable for making tamper resistant pharmaceutical composition because of limitation of its physicochemical properties.

The problem underlying the present invention is which crucial property of water soluble ingredient must required to inhibit extraction of the drug substance form pharmaceutical composition by hot water having temperature greater than about 90° C. and thereby creating difficulties in getting pure drug substance from the dosage form. Inventors found that the cloud point of water soluble ingredient is crucial property to inhibit separation of drug substance from the water soluble ingredient by hot water having temperature greater than about 90° C. and thereby create difficulties in getting pure drug substance from the dosage form for abuse.

In preferred embodiment, a tamper resistant pharmaceutical composition comprising
a. drug substance;
b. water soluble ingredient having cloud point greater than about 90° C.; and
c. optionally at least one pharmaceutically acceptable ingredient;
wherein the cloud point of the water soluble ingredient inhibit extraction of the drug substance from the pharmaceutical composition by hot water having temperature greater than about 90° C.

In preferred embodiment, a tamper resistant pharmaceutical composition comprising
a. drug substance;
b. water soluble ingredient having cloud point lower than about 90° C.;
c. cloud point modifier in an amount sufficient to increase cloud point of the water soluble ingredient till at least about 90° C.; and
d. optionally at least one pharmaceutically acceptable ingredient;
wherein the water soluble ingredient and the cloud point modifier inhibit extraction of the drug substance from the pharmaceutical composition by hot water having temperature greater than about 90° C.

In preferred embodiment, tamper resistant pharmaceutical composition of the present invention reduce or mitigate potential abuse by making it more difficult for the pharmaceutical active ingredient to be extracted from the dosage form by hot water having temperature greater than about 90° C. and used or administered in a manner other than that originally intended.

In certain embodiment, tamper resistant pharmaceutical composition according to the present invention is prepared by non-thermal process or thermal process. In thermal process, heat is employed in at least one stage of manufacturing process of pharmaceutical dosage form while heat is not employed in non thermal process during manufacturing process of pharmaceutical dosage form.

In one embodiment, tamper resistant pharmaceutical composition prepared by thermal process exhibit higher breaking strength compare to prepared by non-thermal process.

### BRIEF DESCRIPTION OF THE DRAWINGS

FIG. 1A, 2A, 3A; FIG. 1B, 2B, 3B and FIG. 3A, 3B, 3C shows photographs of extraction study of example 5 while FIG. 4 show graphical representation of % drug extracted from the dosage form comprising different water soluble ingredient by water at room temperature and after application of heat to achieve temperature greater than about 90° C.

FIG. 1A is a photograph that depicts a side view of the content of M1 beaker having tablet of example 2 with 25 ml of purified water as mentioned in example 5 at t=0, wherein the tablet comprising Hydroxypropyl cellulose (Klucel HXF) as water soluble ingredient having cloud point lower than 90° C.

FIG. 2A is a photograph that depicts a side view of the content of M2 beaker having tablet of example 3 with 25 ml of purified water as mentioned in example 5 at t=0, wherein the tablet comprising Hydroxypropylmethyl cellulose (Hypromellose K100M) as water soluble ingredient having cloud point lower than 90° C.

FIG. 3A is a photograph that depicts a side view of the content of M3 beaker having tablet of example 4 with 25 ml of purified water as mentioned in example 5 at t=0, wherein the tablet comprising Polyethylene oxide (Polyox WSR 303-Leo) as water soluble ingredient having cloud point greater than 90° C.

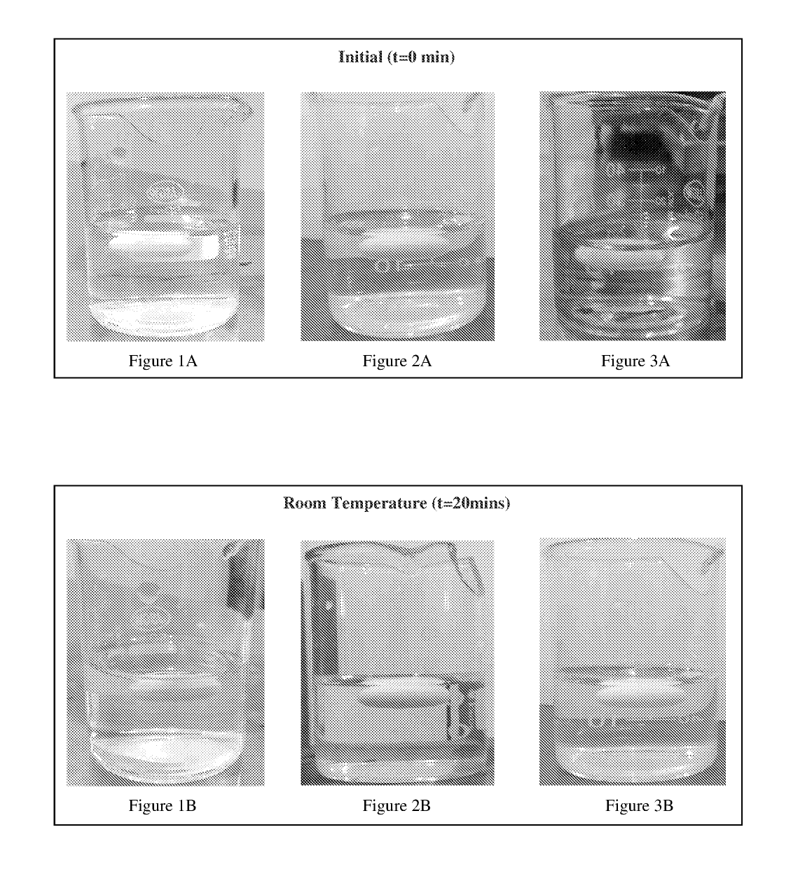

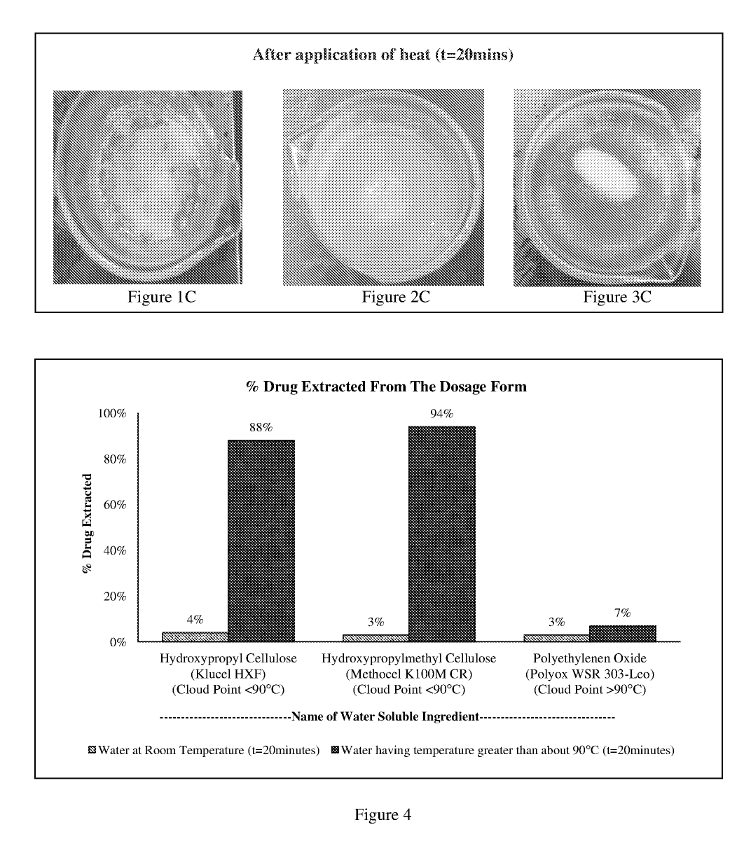

FIG. 1B is a photograph that depicts a side view of the content of M1 beaker having tablet of example 2 with 25 ml of purified water after 20 minutes (t=20 minutes) at room temperature as mentioned in example 5, wherein the tablet comprising Hydroxypropyl cellulose (Klucel HXF) as water soluble ingredient having cloud point lower than 90° C. was remain intact at room temperature after t=20 minutes.

FIG. 2B is a photograph that depicts a side view of the content of M2 beaker having tablet of example 3 with 25 ml of purified water after 20 minutes (t=20 minutes) at room temperature as mentioned in example 5, wherein the tablet comprising Hydroxypropylmethyl cellulose (Hypromellose K100M) as water soluble ingredient having cloud point lower than 90° C. was remain intact at room temperature after t=20 minutes.

FIG. 3B is a photograph that depicts a side view of the content of M3 beaker having tablet of example 4 with 25 ml of purified water after 20 minutes (t=20 minutes) at room temperature as mentioned in example 5, wherein the tablet comprising Polyethylene oxide (Polyox WSR 303) as water soluble ingredient having cloud point greater than 90° C. was remain intact at room temperature after t=20 minutes.

FIG. 1C is a photograph that depicts a top view of the content of M1 beaker having tablet of example 2 with 25 ml of purified water after 20 minutes (t=20 minutes) of heating as mentioned in example 5, wherein the tablet comprising Hydroxypropyl cellulose (Klucel HXF) as water soluble ingredient having cloud point lower than 90° C. was disintegrate into very small particles.

FIG. 2C is a photograph that depicts a top view of the content of M2 beaker having tablet of example 3 with 25 ml of purified water after 20 minutes (t=20 minutes) of heating as mentioned in example 5, wherein the tablet comprising Hydroxypropylmethyl cellulose (Hypromellose K100M) as water soluble ingredient having cloud point lower than 90° C. was disintegrate into very small particles.

FIG. 3C is a photograph that depicts a top view of the content of M3 beaker having tablet of example 4 with 25 ml of purified water after 20 minutes (t=20 minutes) of heating as mentioned in example 5, wherein the tablet comprising Polyethylene oxide (Polyox WSR 303-Leo) as water soluble ingredient having cloud point greater than 90° C. was still floated on top surface of boiled purified water even after 20 minute of heating.

FIG. 4 show the impact of application of heat on extraction process of the drug substance from the pharmaceutical composition comprising water soluble ingredient namely hydroxypropyl cellulose (Klucel HXF), hydroxypropylmethyl cellulose (Methocel K100M) and Polyethylene oxide (Polyox WSR303-Leo) having cloud point <90° C., <90° C. and >90° C., respectively, by water.

### DETAILED DESCRIPTION OF THE INVENTION

The term “abuse” is intended to denote the use of a drug or drugs in order to induce euphoria, i.e. the use is not intended to cure a disease or alleviate disease symptoms, but rather for obtaining intoxication.

According to an invention, a tamper resistant pharmaceutical composition releases the drug or drugs in at least one of immediate release form and in controlled release form. The term “controlled release” is used in present invention to refer “delayed release”, “extended release”, “prolong release”, “long-acting”, “modified release”, “slow release”, “sustained release”, “pulsatile release”, “time release” and the like, all of which are understood to refer to a release which is later or slower than “immediate release” or “release at any rate after certain period of time.”

Many pharmaceutical products are sometimes the subject of abuse. There are various routes of administration an abuser may commonly employ to abuse drug formulation. The most common administration methods of drug abuse include 1) parenteral (e.g. intravenous injection) after extraction of drug from pharmaceutical dosage form, 2) intranasal (e.g., snorting) after tampering pharmaceutical dosage form, and 3) oral ingestion of controlled release pharmaceutical dosage form after tampering of dosage form. Because the potential for abuse of prescription drug products has become an important issue for the world, the pharmaceutical industry is striving to develop tamper resistant pharmaceutical formulations in order to reduce the potential for misuse of prescription drugs.

Different pharmaceutical companies adopt different technologies for making tamper resistant pharmaceutical dosage form such as (1) addition of water soluble gelling agent, wherein the gelling agent form a gel upon contact with aqueous media which create difficulties in abusing via parenteral route; (2) making pharmaceutical dosage form crush resistant using thermoplastic ingredients that resists pulverization of dosage form by application of force with conventional means which are conventionally available to an abuser (such as for example a mortar and pestle, a hammer, spoon or other usual means for pulverization); (3) addition of antagonist in non releasable form in pharmaceutical dosage form comprising drug susceptible to abuse, wherein said non releasable antagonist release only upon tampering of pharmaceutical dosage form; (4) addition of nasal irritating excipients in pharmaceutical dosage form that resist abuse via nasal route; and like.

Majority of drug substances having abuse potentiality are water soluble. Tamper resistant pharmaceutical composition comprising drug substance along with water soluble ingredient adds difficulties in extraction of the drug substance from the composition. So it creates complexity in getting pure drug substance from the dosage form without extraction step for the purpose of abuse via parenteral or nasal administration.

During extraction study of the drug substance from the pharmaceutical composition, inventors surprisingly found that all kind of water soluble ingredients are not suitable for making tamper resistant pharmaceutical composition because of limitation of its physicochemical properties and thereby it is easy to separate the drug substance form the water soluble ingredient.

The problem underlying the present invention is which crucial property of water soluble ingredient must required to inhibit extraction of drug substance form pharmaceutical composition by hot water having temperature greater than about 90° C. and thereby creating difficulties in getting pure drug substance from dosage form. During checking an intensity of abuse deterrent property of dosage form comprising different water soluble ingredients by extraction process using hot water having temperature greater than about 90° C., i.e. separation of drug substance from water soluble ingredient, inventor surprisingly found that all kind of water soluble ingredients are not suitable for making tamper resistant pharmaceutical composition because of limitation of its physicochemical properties and thereby it is easy to separate drug substance form water soluble ingredient. Inventors found that the cloud point of the water soluble ingredient is crucial property which inhibits extraction of drug substance from water soluble gelling agent by hot water having temperature greater than about 90° C. and thereby create difficulties in getting pure drug substance for abuse. Impact of the cloud point of different water soluble ingredients used in making of dosage form on extraction of drug substance from the dosage form is further disclosed in examples of the present invention.

A well known water soluble ingredient is hydroxypropylmethyl cellulose or methylhydroxypropyl cellulose (hypromellose), which is also used as gelling polymer in making of tamper resistant pharmaceutical dosage form. Hypromellose (HPMC K100M) is water soluble gel forming polymer and having very high viscosity about 100,000 cp of 2% solution. Cloud point of the hypromellose is about 70° C. In other word, the hypromellose starts to precipitate from gel state or solution state at a temperature above 70° C. So, for abuser, it is easy to separate drug substance from tamper resistant pharmaceutical dosage form if said dosage form prepared using the hypromellose as gelling agent, because abuser first dissolve the dosage form in water, wherein the hypromellose form a mixture of gel upon contact with water which is difficult to abuse as such via parenteral, however when abuser heat the gel mixture to the temperature above cloud point of the hypromellose (greater than 70° C.), said hypromellose precipitate out from gel mixture and release entrapped drug in water, which is easy to filter and separate drug from the hypromellose or from the dosage form using conventional filtration process. This indicates that the cloud point of hypromellose is limiting characteristic to use hypromellose as water soluble ingredient in tamper resistant composition. In other word, hypromellose is not suitable to use as water soluble ingredient in tamper resistant dosage form because it is easy to separate the drug substance from the hypromellose as water soluble ingredient due to its lower cloud point.

To the best of our knowledge, existing prior art does not teach about which crucial property of water soluble ingredient must required to inhibit extraction of the drug substance form the pharmaceutical composition by hot water having temperature greater than about 90° C.

In preferred embodiment, a tamper resistant pharmaceutical composition comprising
a. drug substance;
b. water soluble ingredient having cloud point greater than about 90° C.; and
c. optionally at least one pharmaceutically acceptable ingredient;
wherein the cloud point of the water soluble ingredient inhibit extraction of the drug substance from the pharmaceutical composition by hot water having temperature greater than about 90° C.

In preferred embodiment, a tamper resistant pharmaceutical composition comprising
a. drug substance;
b. water soluble ingredient, wherein cloud point of the water soluble ingredient is greater than about 90° C.;
c. optionally cloud point modifier in an amount sufficient to increase cloud point of the water soluble ingredient till at least about 100° C.; and
d. optionally at least one pharmaceutically acceptable ingredient;
wherein the drug substance produce an intended therapeutic effectiveness in a subject in need thereof after correct administration of a unit dosage form of the pharmaceutical composition;
wherein the water soluble ingredient inhibit extraction of the drug substance from the pharmaceutical composition by hot water having temperature greater than about 90° C., wherein quantity of the hot water is at least sufficient to dissolve all amount of the drug substance of the unit dosage form.

In preferred embodiment, a tamper resistant pharmaceutical composition comprising
a. drug substance;
b. water soluble ingredient having cloud point lower than about 90° C.;
c. cloud point modifier in an amount sufficient to increase cloud point of the water soluble ingredient till at least about 90° C.; and
d. optionally at least one pharmaceutically acceptable ingredient;
wherein the water soluble ingredient and the cloud point modifier inhibit extraction of the drug substance from the pharmaceutical composition by hot water having temperature greater than about 90° C.

In preferred embodiment, a tamper resistant pharmaceutical composition comprising
a. drug substance;
b. water soluble ingredient having cloud point lower than about 90° C.;
c. cloud point modifier in an amount sufficient to increase cloud point of the water soluble ingredient till at least about 90° C.; and
d. optionally at least one pharmaceutically acceptable ingredient;
wherein the drug substance produce an intended therapeutic effectiveness in a subject in need thereof after correct administration of a unit dosage form of the pharmaceutical composition;
wherein the water soluble ingredient and the cloud point modifier inhibit extraction of the drug substance from the pharmaceutical composition by hot water having temperature greater than about 90° C., wherein quantity of the hot water is at least sufficient to dissolve all amount of the drug substance of the unit dosage form.

The term “water soluble ingredient” refers to ingredient which is soluble in water at all pH or soluble in water at certain pH or soluble in presence of other excipients.

The term “cloud point” refers to “the temperature at which (or above which) water soluble ingredient start to precipitate from solution state or gel state” or “a temperature above which clear solution or gel comprising water soluble ingredient start to become cloudy or turbid”. In present invention, cloud point is particularly discussed with respect to the water.

The term “cloud point modifier” refers to a compound which influences a cloud point of the water soluble ingredient. In particular, the cloud point modifier raise cloud point of the water soluble ingredient.

The term “unit dosage form” refers to intact unit of pharmaceutical composition comprising the therapeutically effective dose of the drug substance suitable for administration to a subject in need thereof. Examples of unit dosage forms include, but are not limited to, tablets, capsules, suppository, lollipops, lozenges and particulates packed in unit dose container or compressed to form tablet. Example of particulates include, but not limited to, microtablets or minitablets, micropellets, granules, spheres, spheroids, beads, pellets, particles, microparticles, nanoparticles and like. In preferred embodiments, the unit dosage form comprises a tablet and capsule.

According to present invention, the standard room temperature is between 20° C. and 26° C., with an average temperature of 23° C.

As per literature, water is starting to evaporate at temperature about 95-100° C. So it would be a very difficult for abuser to separate drug substance from water soluble ingredient having cloud point greater than about 90° C. by water having temperature greater than about 90° C., this is because evaporation of the water is start at around 95-100° C. and it is very difficult for abuser to maintain temperature between 90-95° C. conventionally without using sophisticated equipments. In addition to this, drug is also starting to degradation at higher temperature in presence of water.

Hence, it was found that tamper resistant pharmaceutical composition comprising water soluble ingredient having cloud point greater than about 90° C. reduce or mitigate potential abuse by making more difficulties during extraction of the drug substance from the pharmaceutical composition by hot water having temperature greater than about 90° C. than other pharmaceutical composition comprising a water soluble ingredient having cloud point lower than about 90° C. without a cloud point modifier.

In certain embodiment, tamper resistant pharmaceutical composition comprising at least one cloud point modifier, wherein the cloud point modifier increase cloud point of the water soluble ingredient till at least about 90° C. In certain embodiment, tamper resistant pharmaceutical composition comprising at least one cloud point modifier, wherein the cloud point modifier increase cloud point of the water soluble ingredient till at least about 100° C.

There are many ingredients which increase cloud points of the water soluble ingredient. For example, surfactant increases the viscosity and the cloud points of water soluble polymer. The interaction between surfactant and water soluble ingredient have been widely investigated and available in the literature. One of such kind of examples also available in book “Hydrophilic Matrix Tablets For Oral Controlled Release”, edited by Peter Timmins, Samuel R. Pygall and Colin D. Melia, on page no. 151-152. An example discloses the study of interaction between Hydroxypropylmethyl Cellulose (HPMC) and anionic surfactant Sodium Dodecyl Sulphate (SDS) in water by Nilsson. Nilsson proposed that SDS adsorbs in cooperative manner as molecular cluster, forming a small micelles, the core of which are able to solubilise the methoxy-rich ‘junction zones’ (hydrophobic region) of the HPMC polymer chain. This increases the polymer solubility and raises the cloud point temperature of polymer.

In preferred embodiment, tamper resistant pharmaceutical composition is manufacture either by non-thermal process or thermal process. In thermal manufacturing process, heat is employed in at least one stage of manufacturing process of the pharmaceutical composition while heat is not employed in non thermal manufacturing process during manufacturing of the pharmaceutical composition.

In pharmaceutical industry, thermal process is well known for making innovative pharmaceutical composition from last few decades. Examples of thermal process include hot melt extrusion (HME), melt granulation, curing of pharmaceutical composition at melting temperature, etc.

In certain embodiment, tamper resistant pharmaceutical composition prepared by thermal process comprising step of exposing the pharmaceutical composition to the temperature which is at least melting or softening temperature of the thermoplastic ingredient used in the pharmaceutical composition. In certain embodiment, water soluble ingredient also possesses thermoplastic property.

In certain embodiment, tamper resistant pharmaceutical composition manufacture by thermal process exhibit higher breaking strength compare to manufacture by non-thermal process. In certain embodiment, the pharmaceutical composition manufactured by thermal process exhibit breaking strength at least sufficient to resist pulverisation of the dosage form and thereby not more than 35% pulverized particles of the pharmaceutical composition pass through #200 sieve (75 microns).

In certain embodiment, the pharmaceutical composition is prepared by first mixing the drug substance with the water soluble ingredient and then shaping to form a unit dosage form.

In certain embodiment, a unit dosage form of the pharmaceutical composition comprising at least two populations of particles with or without coating of water insoluble ingredient, wherein a first population of particles comprises the drug substance and a second population of particles comprises the water soluble ingredient. The term “particles” is used generally to refer to individual, discrete particles, irrespective of their size, shape or morphology. In present invention, the term “particles” includes, but not to be limited, pellets, beads, granules, spheroids, minitablets or microtablets. Water insoluble ingredients are those ingredients which are insoluble in water at all pH or insoluble at certain pH.

According to present invention, tamper resistant pharmaceutical composition can be formulated in form of, but not limited to, tablet, microtablets or minitablets, micropellets, granules, spheroids, beads or pellets, filled in capsule, powders, caplets, pills, suppositories, liquid, gels and like.

In certain embodiments, the present invention is directed to a method of treating a disease condition of a subject comprising administering a unit dosage form of the tamper resistant pharmaceutical composition to the subject in need thereof. The methods of treating a disease condition include single or repeated dosing over a time interval.

As used herein, the term “about” is understood to mean ±10% of the value referenced. For example, “about 45” is understood to literally mean 40.5% to 49.5%.

### Components of Tamper Resistant Pharmaceutical Composition:

### Drug Substance:

In preferred embodiment, a tamper resistant pharmaceutical composition comprises at least one drug substance.

The terms “drug”, “active substance”, “active ingredient”, “active agent”, “drug substance”, “pharmacologically active agent” and “physiologically active agent” or “drug substance in physiologically active form” are used interchangeably herein to refer to a chemical compound that induces a desired pharmacological or physiological effect or biological activity. The terms also encompass pharmaceutically acceptable derivatives of those active agents specifically mentioned herein, including, but not limited to, salts, solvates, polymorphs, hydrates, complexes with one or more molecules, prodrug, active metabolites, analogs, and the like. When the terms “drug”, “active agent”, “pharmacologically active agent” and “physiologically active agent” or “drug substance in physiologically active form” are used, or when a particular drug, for example oxycodone, is identified, it is to be understood as including the active agent as well as pharmaceutically acceptable salts, solvates, polymorphs, hydrates, complexes with one or more molecules, prodrugs, active metabolites, homologue and analogs.

In some embodiment, the drug substance is added in dosage form in powder form, in amorphous form, in crystalline form, in micronized form, in complexes with one or more molecules, in combination of two or more active substance or any combination of thereof.

In some embodiments, the active ingredient include, but are not limited to analgesics, anti-inflammatory agents, anti-helminthics, anti-arrhythmic agents, anti-asthma agents, anti-bacterial agents, anti-viral agents, anti-coagulants, anti-dementia agents, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, immunosuppressants, antiprotozoal agents, anti-thyroid agents, anti-tussives, anxiolytics, sedatives, hypnotics, neuroleptics, neuroprotective agents, β-blockers, cardie inotropic agents, cell adhesion inhibitors, corticosteroids, cytokine receptor activity modulators, diuretics, anti-Parkinson's agents, gastro-intestinal agents, histamine H-receptor antagonists, keratolyses, lipid regulating agents, muscle relaxants, nitrates and other anti-anginal agents, non-steroid anti-asthma agents, nutritional agents, opioid analgesics, sex hormones, stimulants and anti-erectile dysfunction agents; and salts, esters, and mixtures thereof.

In preferred embodiments, an active drug substance is associated with abuse syndromes and the active drug substance may, thus for example, be selected from opioids, opiates, CNS depressants or sedative, anxiolytics, narcotic, tranquilizers, barbiturates, hormones, CNS stimulants, cannabinoids, nicotine-like compounds, glutamate antagonists, N-methyl-D-aspartate (NMDA) antagonists or drugs that can cause psychological and/or physical dependence. Drugs that are preferred include those classified as Schedule I, II, III, IV and V drugs based upon the substance's medicinal value, harmfulness, and potential for abuse or addiction under the control substance act.

Tamper resistant pharmaceutical composition according to the invention is very particularly suitable for preventing abuse of active drug substances includes, but not limited to, are:

1-(1-Phenylcyclohexyl)pyrrolidine, 1-(2-Phenylethyl)-4-phenyl-4-acetoxypiperidine, 1-[1-(2-Thienyl)-cyclohexyl]piperidine, 1-[1-(2-Thienyl)cyclohexyl]pyrrolidine, 1-Methyl-4-phenyl-4-propionoxy-piperidine, 1-Phenylcyclohexylamine, 1-Piperidinocyclohexanecarbonitrile, 2,5-Dimethoxy-4-ethylamphetamine, 2,5-Dimethoxyamphetamine, 2C-B-(4-bromo-2,5-dimethoxypenethylamine), 2C-D (2,5-dimethoxy-4-methylphenethylamine), 2C-I (4-iodo-2,5-dimethoxy-phenethylamine), 2C-T-2 (2,5-dimethoxy-4-ethylthiophenethylamine), 2C-T-4 (2,5-dimethoxy-4-isopropyl thiophenethylamine), 2C-T-7 (2,5-dimethoxy-4-(n)-propylthiopenethylamine), 3,4-Methylene-dioxymethamphetamine, 3,4,5-Trimethoxyamphetamine, 3,4-Methylenedioxyamphetamine, 3,4-Methylenedioxy-N-ethylamphetamine, 3-Methylfentanyl, 3-Methylthiofentanyl, 4-Bromo-2,5-dimethoxyamphetamine, 4-Bromo-2,5-dimethoxyphenethylamine, 4-Methoxyamphetamine, 4-Methyl-2,5-dimethoxyamphetamine, 4-Methylaminorex (cis isomer), 5-MeO-DMT (5-Methoxy-N,N-diisopropyltryptamine), 5-MeO-DMT (5-Methoxy-N,N-dimethyltryptamine), 5-Methoxy-3,4-methylenedioxyamphetamine, Acetorphin, Acetorphine, Acetyl-alpha-methylfentanyl, Acetyl-alpha-methylfentanyl, Acetyldihydrocodeine, Acetylmethadol, Acetylmethadol, aniloridine, Alfentanil, Allobarbital, Allylprodin, Allylprodine, Alphacetylmethadol except levo-alphacetylmethadol, Alpha-ethyltryptamine, Alphameprodine, Alphamethadol, Alphamethadol, Alpha-Methylfentanyl, Alpha-Methylthiofentanyl, Alphaprodine, Alprazolam, Amfepramon, Amfetaminil, Amineptin, Aminorex, Amobarbital, amfepramone, Amphetamine, amphetaminil, AmyInitrit (all isomers of the amyl group), Anabolic steroids, Anileridine, apocodeine, Aprobarbital, Barbital, Barbituric acid derivative, BDB (3,4-methylenedioxyphenyl)-2-butanamine), Benzethidin, Benzethidine, Benzoylecgonine, Benzphetamine, Benzphetamine, Benzylmethylketon, Benzylmorphine, Betacetylmethadol, Beta-Hydroxy-3-methylfentanyl, Beta-Hydroxyfentanyl, Betameprodine, Betameprodine, Betamethadol, Betaprodine, Bezitramide, Boldenone, Brolamfetamin, Bromazepam, Brotizolam, Bufotenine, Buprenorphine, Butabarbital, Butalbital, Butobarbital, Butorphanol, BZP (A 2)(1-benzylpiperazin), Camazepam, Cannabis, Carfentanil, carfentanyl, Catha edulis, Cathine, Cathinone, Chloral betaine, Chloral hydrate, Chlordiazepoxide, Chlorhexadol, Chlorotestosterone (same as clostebol), Chlorphentermine, Clobazam, Clonazepam, Clonitazene, Clorazepate, Clortermine, Clostebol, Clotiazepam, Cloxazolam, Coca Leaves, Cocaine, Codeine, Codeine & isoquinoline alkaloid, Codeine methylbromide, Codeine-N-oxide, Codoxim, cyclorphan, Cyclobarbital (Hexemal NFN), cyclazocine, Cyprenorphine, Dehydrochlormethyltestosterone, Delorazepam, Desomorphine, Dexamfetamine, dexamphetamine, dexmethylphenidate, Dexfenfluramine, Dextromoramide, dextromethorphan, Dextropropoxyphene, dezocine, Diacetylmorphine, diamorphone, Diampromide, diapromide, Diazepam, Dichloralphenazone, Diethylpropion, Diethylthiambutene, Diethyltryptamine, Difenoxin, Dihydrocodeine, Dihydroetorphine, Dihydromorphine, Dihydrotestosterone, dimephetanol, Dimenoxadol, Dimepheptanol, Dimethylthiambutene, Dimethyltryptamine, Dioxaphetyl butyrate, Diphenoxylate, Dipipanone, Diprenorphine, Dronabinol, Drostanolone, Drotebanol, Ecgonine, eptazocine, Estazolam, Ethchlorvynol, Ethinamate, ethoheptazine, Ethyl loflazepate, Ethylestrenol, Ethylmethylthiambutene, Ethylmorphine, Eticyclidin, Etilamfetamine, Etonitazene, Etorphine, Etoxeridine, Etryptamine, Fencamfamin, Fenethylline, Fenetylline, Fenfluramine, Fenproporex, Fentanyl, Fludiazepam, Flunitrazepam, Fluoxymesterone, Flurazepam, Formebolone, Fungi and Spores of the species Psilocype Semilanceata, Furethidine, Gammahydroxybutanic acid, Glutethimide, Halazepam, Hallucinogens, Haloxazolam, Heroine, Hydrocodone, Hydrocodone & isoquinoline alkaloid, Hydromorphinol, Hydromorphone, Hydroxypethidine, Ibogaine, Isobutylnitrit, Isomethadone, Ketamine, Ketazolam, Ketobemidone, lefetamine, Levamfetamine, levallorphan, Levo-alphacetylmethadol, Levo-methamphetamine, Levomethorphan, Levomoramide, Levophenacylmorphan, Levorphanol, lisdexamphetamine, lofentanil, loperamide, Loprazolam, Lorazepam, Lormetazepam, Lysergic acid, Lysergic acid amide, Lysergic acid diethylamide, Marijuana, Mazindol, MBDN(N-methyl-1-(3,4-methylenedioxyphenyl)-2-butanamine), mCPP (1-(3-chlorphenyl)piperazine), Mebutamate, Mecloqualone, Medazepam, Mefenorex, MeOPP (1-(4-methoxyphenyl)piperazine), Meperidine intermediate, Meprobamate, meptazinol, Mescaline, Mesocarb, Mesterolone, Metamfetamine, Metazocine, Methadone, Methadone intermediate, Methamphetamine, Methandienone, Methandranone, Methandriol, Methandrostenolone, Methaqualone, Methcathinone, Methenolone, Methohexital, Methyldesorphine, Methyldihydromorphine, Methylphenidate, Methylphenobarbital (mephobarbital), Methyltestosterone, Methyprylone, Metopone, Mibolerone, Midazolam, Modafinil, Moramide-intermediate, Morpheridine, Morphine, morphine 3-glucuronide, morphine 6-glucuronide, Morphine methylbromide, Morphine methylsulfonate, Morphine-N-oxide, Myrophine, N,N-Dimethylamphetamine, Nabilone, nalbuphine, Nalorphine, Nandrolone, narccine, N-Ethyl-1-phenylcyclohexylamine, N-Ethyl-3-piperidyl benzilate, N-Ethylamphetamine, N-Hydroxy-3,4-methylenedioxyamphetamine, Nicocodeine, Nicocodine, Nicodicodine, Nicomorphine, Nimetazepam, Nitrazepam, N-Methyl-3-piperidyl benzilate, Noracymethadol, Norcodeine, Nordiazepam, Norethandrolone, Norlevorphanol, Normethadone, Normorphine, Norpipanone, ohmefentanyl, Opium, Oxandrolone, Oxazepam, Oxazolam, oxycodeine, Oxycodone, Oxymesterone, Oxymetholone, Oxymorphone, papavereturn, Para-Fluorofentanyl, Parahexyl, Paraldehyde, pethidine, Pemoline, Pentazocine, Pentobarbital, Petrichloral, Peyote, Phenadoxone, Phenampromide, Phenazocine, Phencyclidine, Phendimetrazine, Phenmetrazine, Phenobarbital, Phenomorphan, Phenoperidine, Phentermine, Phenylacetone, Pholcodine, Piminodine, Pinazepam, Pipradrole, Piritramide, PMMA (paramethyxymethyl amphetamine), Prazepam, prodine, Proheptazine, promedol, Properidine, Propiram, propheptazine, propoxyphene, Psilocybine, Psilocyn, Pyrovalerone, Quazepam, Racemethorphane, Racemoramide, Racemorphane, Remifentanil, Salvia divinorum, Salvinorin A, Secobarbital, Secobarbital, Sibutramine, SPA, Stanolone, Stanozolol, Sufentanil, Sulfondiethylmethane, Sulfonethylmethane, Sulfonmethane, Talbutal, Tapentadol, Temazepam, Tenamfetamin, Testolactone, Testosterone, Tetrahydrocannabinols, Tetrazepam, TFMPP (1-(3-triflourmethylphenyl)piperazine), Thebacon, Thebaine, Thiamylal, Thiofentanyl, Thiopental, Tiletamine, Tramadol, Zolazepam, Zolazepam, Tilidine, Trenbolone, Triazolam, Trimeperidine, Vinbarbital, Zaleplon, Zipeprol, Zolpidem, Zopiclon.

In one embodiment of present invention, the drug substance may also include new chemical entity for which the amount of information is limited. In such cases, the dosage form regimen needs to evaluate based on preclinical and clinical trials.

In some embodiments, a dosage form contains therapeutically effective dose of drug substance to provide a therapeutic effectiveness in a subject in need thereof.

The above mentioned active drug substances may also be in the form of pharmaceutically acceptable salts, uncharged or charged molecules, molecular complexes, solvates or anhydrates thereof, and, if relevant, isomers, enantiomers, racemic mixtures, and mixture thereof.

### Water Soluble Ingredients:

In certain embodiment, tamper resistant pharmaceutical composition comprises at least one water soluble ingredient. In certain embodiment, tamper resistant pharmaceutical composition comprises at least one water soluble ingredient having cloud point greater than about 90° C. In certain embodiment, tamper resistant pharmaceutical composition comprises at least one water soluble ingredient having cloud point lower than about 90° C.

Suitable water soluble ingredient may be natural, semi-synthetic or synthetic. Suitable water soluble ingredient may selected from group consisting of, but not limited to, polymer, sugar, salts, salts of organic acid, water soluble dye, water soluble irritating agent, acid, base and polysaccharide.

Suitable polymer may be natural, semi-synthetic or synthetic. In some embodiment, water soluble polymer include polyalkylene oxide such as polyethylene oxide (PEO), polypropylene oxide, combinations thereof, or copolymers thereof; cellulose ethers include hydroxypropyl cellulose (HPC), hydroxypropyl methylcellulose (HPMC), carboxymethylcellulose (CMC), methylcellulose, hydroxyethyl cellulose (HEC), hydroxyethyl methylcellulose, ethylhydroxyethylcellulose, ethylmethylcellulose, hydroxymethylcellulose, and like; polyalkylene glycol such as polyethylene glycol; poloxamer; polysaccharides include, without limit, celluloses, starches, pectins, chitins, gums; gums include xanthan gum, acacia gum, diutan gum, gellan gum, guar gum, fenugreek gum, locust bean gum, pullulan, welan gum, or combinations thereof; polycarboxylic acid such as polyacrylic acid, polyacrylic acid-co-acrylamide, polymethacrylate, polyhydroxyethyl methacrylate, combinations, or copolymers thereof; polyamine such as polyethyleneimine, polyvinylamine; polypeptide such as gelatin, albumin, polylysine, soy protein; polyolefinic alcohol (such as polyvinyl alcohol), or a polyvinyl lactam such as, e.g., polyvinylpyrrolidone, polyvinyl caprolactam; alginic acid and its derivative; methacrylica acid and its copolymer; Polyacrylic acid and copolymer thereof; and like.

Preferred sugars include dextrose, glucose, arabinose, ribose, arabinose, xylose, lyxose, xylol, allose, altrose, inositol, glucose, sorbitol, mannose, gulose, Glycerol, idose, galactose, talose, trehalose, mannitol, erythritol, ribitol, xylitol, maltitol, isomalt, lactitol, sucrose, raffinose, maltose, fructose, lactose, dextrin, dextran, amylase and xylan. Water soluble salts to modify osmolarity include sodium or potassium chloride, calcium chloride or magnesium chloride, lithium chloride, lithium, sodium or potassium hydrogen phosphate, lithium, sodium or potassium dihydrogen phosphate.

Preferred acids include ascorbic acid, 2-benzene carboxylic acid, benzoic acid, fumaric acid, citric acid, maleic acid, serbacic acid, sorbic acid, edipic acid, edetic acid, glutamic acid, toluene sulfonic acid, water-soluble amino acids such as glycine, leucine, alanine, or methionine and tartaric acid; and like. Salts of organic acid include sodium or potassium acetate, magnesium succinate, sodium benzoate, sodium citrate, sodium ascorbate and like.

Preferred base include magnesium oxide, meglumine, sodium oxide, sodium hydroxide, sodium bicarbonate, sodium potassium tartrate, bismuth aluminate, bismuth carbonate, bismuth subcarbonate, bismuth subgallate, bismuth subnitrate, potassium citrate, sodium citrate, sodium carbonate, potassium bicarbonate, potassium carbonate, calcium carbonate, calcium phosphate, dibasic calcium phosphate, dihydroxyaluminum aminoacetate, dihydroxyaluminum sodium carbonate, glycine, magnesium glycinate, magnesium hydroxide, magnesium carbonate, sodium borate, aluminum oxide, aluminum hydroxide, ammonium Carbonate, monoethanolamine, diethanolamine, triethanolamine, Potassium Hydroxide, Sodium Phosphate Dibasic, Trolamine, sodium potassium tartrate, tribasic sodium phosphate, tricalcium phosphate and like.

The term “irritating agent” means any agent that produce irritation upon administration via parenteral route or nasal route. Irritating agent also include burning agent or flushing agent. Suitable compounds are believed to be within the knowledge of a practitioner skilled in the relevant art, and can be found in the Handbook of Pharmaceutical excipients, 7th edition, the entire content of which is hereby incorporated by reference.

Preferred dye includes D&C dye/lake, FD&C dye, an FD&C lake, caramel, ferric oxide, a natural coloring agent, and a combination thereof. The amount of dye used will vary as desired. Preferably the composition is non-toxic, edible, color stable in light and air and free of potential hazards to human health.

In certain embodiment, tamper resistant pharmaceutical composition comprises at least one water soluble ingredient having cloud point greater than about 90° C.

In preferred embodiment, preferred water soluble ingredients having cloud point greater than about 90° C. may include, but not limited to, natural, semi-synthetic or synthetic gum or polymer, polyalkylene oxide such as polyethylene oxide, cellulose derivatives such as hydroxyethyl cellulose, polyacrylic acid and copolymer thereof, carboxymethylcellulose (CMC), acid, base, irritating agent, dye, osmotic agent and like. Water soluble ingredient having cloud point greater than about 90° C. further include all ingredients which remains in dissolved state in boiled water.

According to literature, there are many high viscosity gelling polymers which can be used in preparation tamper resistant pharmaceutical composition. Hypromellose K100M is well known example of high viscosity water soluble gelling polymer used in preparation of tamper resistant pharmaceutical composition as gelling polymer. Viscosity of hypromellose K100M is about 100000 cp of 2% solution. However, according to present invention, hypromellose K100M is not suitable polymer for making tamper resistant pharmaceutical composition because lower cloud point (Cloud point of hypromellose K100M is about 70° C.).

Hence, it was found that tamper resistant pharmaceutical composition comprising water soluble polymer having cloud point greater than about 90° C. reduce or mitigate potential abuse by making it more difficult for the pharmaceutical active ingredient to be extracted from the dosage form by hot water having temperature greater than about 90° C. compared to the pharmaceutical composition comprising water soluble polymer having cloud point lower than about 90° C.

A preferred amount of water soluble ingredient is about 0.1% w/w, preferable about more than about 1% w/w, more preferably about more than about 5% w/w and most preferably about more than about 10% w/w of total weight of dosage form or drug substance. A tamper resistant pharmaceutical composition prepared by thermal process comprising water soluble ingredient at least about 10% w/w, preferable at least about 20% w/w and more preferable at least about 30% w/w of total weight of dosage form.

### Cloud Point Modifier:

In certain embodiment, the pharmaceutical composition comprising in addition to drug substance, water soluble ingredients having cloud point lower than about 90° C. and cloud point modifier, wherein the cloud point modifier is present in the composition in an amount sufficient to increase a cloud point of the water soluble ingredient till at least about 90° C.

The term “cloud point modifier” refers to a compound which influences the cloud point of water soluble ingredient. In particular, the cloud point modifiers raise the cloud point of water soluble ingredient.

In one embodiment, cloud point modifier may be selected from group consisting of, but not limited to, surfactant, phospholipids, ionic or non-ionic ingredients, glycols, glycol ethers, electrolytes and like. Suitable cloud point modifier may include, but not limited to, sodium dodecyl sulfate (SDS) or sodium lauryl sulfate (SLS), dodecyltrimethylammonium bromide (DTAB), tetradecyl trimethyl ammonium bromide (TTAB), taurodeoxycholate, glycols, glycol ethers, polyethylene glycols, propylene glycol, cetrimide, dimyristoyl phosphatidyl glycerol, cardiolipin, dimyristoylphosphatidylserine, capronic acid, caprylic acid, dioctylsulfosuccinate (DOSS), sodium oleate, hydroxypropylcyclodextrin, and like. A preferred cloud point modifier is sodium dodecyl sulfate (SDS) or sodium lauryl sulfate (SLS).

In certain embodiment, drug substance itself act as cloud point modifier.

The cloud point modifier is present in the compositions of the present invention in an amount sufficient to raise the cloud point of water soluble ingredient till at least about 90° C. A preferred amount of cloud point modifier is from about 0.1% to 40% w/w of total weight of water soluble ingredient or total weight of dosage form. A more preferred amount of cloud point modifier is from 5% to 25% of total weight of water soluble ingredient or total weight of dosage form.

### Pharmaceutically Acceptable Ingredient

The pharmaceutical composition can also optionally include other ingredients to enhance dosage form manufacture from a pharmaceutical composition of the present invention and/or alter the release profile of drug from pharmaceutical composition of the present invention.

Some embodiment of present invention comprises controlled release ingredient include, but not limited to, natural, synthetic or semi-synthetic polymer, cellulose derivatives, polymethacrylate or methacrylic acid derivatives, vinyl pyrrolidone and copolymers of vinyl pyrrolidone, polyalkylene oxide and copolymer thereof, polyacrylic acid, gums of plant, animal, mineral or synthetic origin, carageenan, polylactic acid or polyglycolic acid and copolymers thereof, polyvinyl alcohol, polyvinyl acetate and its copolymer, fatty acids, wax, fatty alcohol, lipid, steryl alcohol, oil, natural and synthetic glycerides, and like.

Some embodiment of present invention comprises one or more fillers or diluents such as cellulose derivatives, mono, di or tri basic calcium phosphate, sugar, starch derivatives, acid or base, and like.

Some embodiment of present invention comprises one or more disintegrant may be highly/rapidly swellable, moderately swellable or slowly swellable such as vinylpyrrolidone polymers such as crospovidon, cellulose and cellulose derivatives, sodium starch glycolate, starch and starch derivatives, resins, and like.

Some embodiment of present invention may also comprise binder, glidant, lubricant, plasticizers, anti-adhesives, surfactants, colorants, flavoring agents, wiking agent or anti oxidant. Examples of such kind of excipients are described in Handbook of Pharmaceutical excipients, 7th edition.

However, choice of other additives typically depends on the chemical and physical properties of the drug, desire drug release profile and design of the dosage form.

In certain embodiment, water soluble ingredient also act as disintegrant, diluents, binder, lubricant, controlled release ingredient, glidant, surfactant or nasal irritating agent.

### EXAMPLES

Certain aspects of the present invention may be better understood as illustrated by the following examples, which are meant by way of illustration and not limitation. Metformin HCl was used as model drug in making of abuse deterrent dosage form in following examples.

### Example 1

According to present invention, Hydroxypropyl Cellulose (HPC), Hydroxypropyhnethyl Cellulose (HPMC) and Polyethylene Oxide (PEO) are water soluble ingredient. Cloud point of above three polymers is shown in following table:

| Sr. No. | Name of Water Soluble Ingredient | Cloud Point | Remarks |
|---|---|---|---|
| 1 | Hydroxypropyl Cellulose | 40° C.-45° C. | Cloud point lower than 90° C. |
| | (Klucel HXF) | | (<90° C.) |
| 2 | Hydroxypropylmethyl Cellulose | ~71° C. | Cloud point lower than 90° C. |
| | (Methocel K100M) | | (<90° C.) |
| 3 | Polyethylene Oxide | ~96° C. | Cloud point greater than 90° C. |
| | (Polyox WSR 303) | | (>90° C.) |

Dosage form was prepared using above three water soluble ingredient and intensity of abuse deterrent property was evaluated by extraction of the drug substance from the dosage form by hot water having temperature greater than about 90° C. in examples 2 to 8.

### Example 2

Tablet was prepared using water soluble ingredient Hydroxypropyl Cellulose (Klucel HXF) having cloud point lower than 90° C. (<90° C.).

### Composition:

| Sr. No | Ingredients Name | Qty/tab (mg) |
|---|---|---|
| 1 | Metformin HCl | 125.00 |
| 2 | Hydroxypropyl Cellulose (Klucel HXF) | 370.00 |
| 3 | Magnesium Stearate | 5.00 |

| | |
|---|---|
| Total Weight of Tablet | 500.00 |

### Manufacturing Process:

1. Metformin HCl and Klucel HXF were sifted through appropriate sieve and mixed for 10 minutes in appropriate size container.
2. Magnesium Stearate was added to Step 1 blend and mixed for another 5 minutes.
3. Step 2 blend was compressed to target weight of tablet.

### Example 3

Tablet was prepared using water soluble ingredient Hydroxypropylmethyl Cellulose (Methocel K100M) having cloud point lower than 90° C. (<90° C.).

### Composition:

| | | Qty/tab |
|---|---|---|
| Sr. No | Ingredients Name | (mg) |
| 1 | Metformin HCl | 125.00 |
| 2 | Hydroxypropylmethyl Cellulose (Methocel K100M) | 370.00 |
| 3 | Magnesium Stearate | 5.00 |

| | |
|---|---|
| Total Weight of Tablet | 500.00 |

### Manufacturing Process:

1. Metformin HCl and Methocel K100M were sifted through appropriate sieve and mixed for 10 minutes in appropriate size container.
2. Magnesium Stearate was added to Step 1 blend and mixed for another 5 minutes.
3. Step 2 blend was compressed to target weight of tablet.

### Example 4

Tablet was prepared using water soluble ingredient Polyethylene Oxide (Polyox WSR 303-Leo) having cloud point greater than about 90° C. (>90° C.).

### Composition:

| Sr. No | Ingredients Name | Qty/tab (mg) |
|---|---|---|
| 1 | Metformin HCl | 125.00 |
| 2 | Polyethylene Oxide (Polyox WSR 303-Leo) | 370.00 |
| 3 | Magnesium Stearate | 5.00 |

| | |
|---|---|
| Total Weight of Tablet | 500.00 |

### Manufacturing Process:

1. Metformin HCl and Polyox WSR 303-Leo were sifted through appropriate sieve and mixed for 10 minutes in appropriate size container.
2. Magnesium Stearate was added to Step 1 blend and mixed for another 5 minutes.
3. Step 2 blend was compressed to target weight of tablet.

### Example 5

This Example investigates the intensity of abuse deterrent property of the compositions when mixed with 25 ml of water at room temperature and after application of heat. The intent is to simulate an attempt to extract the active ingredient from the water soluble ingredient or the respective dosage form prepared as per example 2, example 3 and example 4.

### Methodology:

The methodology employed was as follows:
1. A tablet of Metformin HCl composition, tablet of example 2, example 3 and example 4, with 25 ml of water were placed in three different glass beakers, respectively, and beakers were marked with M1, M2 and M3 for tablets of example 2, example 3 and example 4, respectively. M1, M2 and M3 beakers were prepared in two sets.
2. One set of M1, M2 and M3 beakers were placed on hot plate and started to heat to achieve temperature greater than about 90° C. while another set of M1, M2 and M3 beaker were placed at room temperature.
3. Photographs of each beaker with contents were taken at t=0 minutes and t=20 minutes.

### Physical Observation:

FIG. 1A, 2A, 3A, FIG. 1B, 2B, 3B and FIG. 1C, 2C, 3C illustrates the physical observation of tablet of Metformin HCl compositions after mixed with 25 ml of water at t=0 minutes at room temperature, t=20 minutes at room temperature and t=20 minutes after application of heat to achieve temperature greater than about 90° C., respectively.

| Composition | M1 Beaker | M2 Beaker | M3 Beaker |
|---|---|---|---|
| Initial | Tablet float on the surface of water in all three beakers. | | |
| (t = 0 min) | (See FIG. 1A, 2A and 3A). | | |
| Room | In all three beakers, tablets remain intact and float on the surface of water, gelling layer | | |
| Temperature | was observed on outer surface of tablet (See FIG. 1B, 2B and 3B). | | |
| (t = 20 mins) | | | |
| After | Tablet was disintegrates into very | Tablet was disintegrates into very | Tablet was remains |
| application | small particles. | small particles. | intact in boiled water |
| of heat | Viscosity of drug solution in | Viscosity of drug solution in | even after 20 minutes and |
| (t = 20 mins) | beaker was very low (almost | beaker was very low (almost | float on the surface of |
| | similar to initial viscosity of water) | similar to initial viscosity of water) | water. |
| | and easy to filter to separate drug | and easy to filter to separate drug | In contrast to M1 and |
| | solution from precipitates of water | solution from precipitates of water | M2 beakers, viscosity of |
| | soluble ingredient by conventional | soluble ingredient by conventional | water in M3 beaker was |
| | filtration process. This is because | filtration process. This is because | increase compared to |
| | after application of heat, water | after application of heat, water | time t = 0 minute and |
| | soluble ingredient lost its gelling | soluble ingredient lost its gelling | compared to M1 and M2 |
| | properties because of its lower | properties because of its lower | beaker at t = 20 minutes |
| | cloud point (below 90° C.) and | cloud point (below 90° C.) and | after application of heat. |
| | precipitated out in water. | precipitated out in water. | See FIG. 3C. |
| | See FIG. 1C. | See FIG. 2C. | It is very difficult to |
| | The aqueous fluid from the | The aqueous fluid from the | separate the drug |
| | resultant drug solution can be | resultant drug solution can be | substance from the |
| | easily removed by evaporation by | easily removed by evaporation by | dosage form composition |
| | application of heat to get pure drug | application of heat to get pure drug | by conventional filtration |
| | substance which is easy to abuse. | substance which is easy to abuse. | process. |

### Example 6

Amount of the drug extracted from the compositions in beaker M1, M2 and M3 of example 5 was as follow:

| Composition | Beaker M1 | Beaker M2 | Beaker M3 |
|---|---|---|---|
| % drug extracted from the | 4 | 3 | 3 |
| dosage form by water at room | | | |
| temperature after 20 minutes | | | |
| % drug extracted from the | 88 | 94 | 7 |
| dosage form by water having | | | |
| temperature greater than about | | | |
| 90° C. after 20 minutes | | | |

Above result clearly shows that the cloud point of the water soluble ingredient play important role in extraction of the drug substance from the dosage form. Dosage form comprising water soluble ingredient having cloud point greater than 90° C. potentially inhibit the extraction of the drug substance from the dosage form while it is easy to extract the drug substance form the dosage form comprising water soluble ingredient having cloud point lower than about 90° C. without cloud point modifier by hot water having temperature greater than about 90° C., in other word, the cloud point of the water soluble ingredient inhibit the extraction of the drug substance form the dosage form.

Above result is graphically presented in FIG. 4 to show the impact of application of heat on extraction process of the drug substance from the pharmaceutical composition comprising water soluble ingredient namely hydroxypropyl cellulose (Klucel HXF), hydroxypropylmethyl cellulose (Methocel K100M) and Polyethylene oxide (Polyox WSR303-Leo) having cloud point <90° C., <90° C. and >90° C., respectively, by water at room temperature and after application of heat.

### Example 7

This Example also investigates the intensity of abuse deterrent property of the compositions after crushing to small particles and then mixed with 25 ml of water at room temperature and at temperature greater than about 90° C. The intent is to simulate an attempt to extract the active ingredient from crushed dosage form prepared as per example 2, example 3 and example 4.

### Methodology:

The methodology employed was as follows:
1. A tablet of Metformin HCl composition of example 2, example 3 and example 4 was crushed to small particles using mortar and pestle.
2. A crushed tablet of Metformin HCl composition of example 2, example 3 and example 4 with 25 ml of water was placed in three different glass beakers marked with M1, M2 and M3 for crushed tablets of example 2, example 3 and example 4, respectively and content of beakers were stirred with glass road for 10 minutes. M1, M2 and M3 beakers were prepared in two sets.
3. One set of M1, M2 and M3 beakers were placed on hot plate and started to heating to achieve temperature greater than about 90° C. while another set of M1, M2 and M3 beaker were placed at room temperature.

### Physical Observation:

Physical observation of crushed tablet of Metformin HCl compositions of example 2, example 3 and example 4 after mixed with 25 ml of water at t=0 minutes at room temperature, t=20 minutes at room temperature and t=20 minutes after application of heat to achieve temperature greater than about 90° C. was as follow:

| Composition | M1 Beaker | M2 Beaker | M3 Beaker |
|---|---|---|---|
| Initial | High viscous gel was observed in all beakers with some undissolved matrix particles of water | | |
| (t = 0 min) | soluble ingredient with entrapped active ingredients. | | |
| | Contents of all three beakers (M1, M2 and M3) were not passed through ASTM #100 sieve. | | |
| Room | After 20 minutes at room temperature, high viscous gel was observed in all beakers with some | | |
| Temperature | undissolved matrix particles of water soluble ingredient with entrapped active ingredients. | | |
| (t = 20 mins) | Contents of all three beakers (M1, M2 and M3) were not passed through ASTM #100 sieve. | | |
| After | Viscous gel was converted | Viscous gel was converted | Viscous gel was remain as |
| application of | into solution state due to | into solution state due to | such with large undissolved |
| heat | precipitation of dissolved | precipitation of dissolved | matrix particles of water |
| (t = 20 mins) | water soluble ingredient. | water soluble ingredient. | soluble ingredient with |
| | Undissolved matrix particles | Undissolved matrix particles | entrapped active ingredients |
| | of water soluble ingredient | of water soluble ingredient | after 20 minutes heating. |
| | with entrapped active | with entrapped active | In contrast to M1 and M2 |
| | ingredients disintegrating into | ingredients disintegrating into | beaker, viscosity of M3 beaker |
| | very small particles. | very small particles. | was increase after 20 minutes |
| | Contents of beaker were | Contents of beaker were | due to more solubilization of |
| | easily passed through ASTM | easily passed through ASTM | undissolved matrix particles |
| | #100 sieve while precipitates | #100 sieve while precipitates | after application of heat. |
| | of the water soluble | of the water soluble | Contents of beaker were not |
| | ingredient were retain on the | ingredient were retain on the | passed through ASTM #100 |
| | sieve. | sieve. | sieve. |

### Example 8

Results of the extraction study of example 5 to example 7 clearly shows that the dosage form comprising water soluble ingredient having cloud point lower than about 90° C. are not potentially inhibit the extraction of the drug substance from the dosage form compared to the dosage form comprising water soluble ingredient having cloud point greater than about 90° C. Following table summarizes the outcome of example 1 to 7.

| | | | % drug extracted | Potentially inhibit the extraction |
|---|---|---|---|---|
| | | | from the dosage form | of the drug substance from the |
| | | | by water having | dosage form by hot water having |
| Sr. | Name of Water Soluble | | temperature greater | temperature greater than about |
| No. | Ingredient of the dosage form | Cloud point | than about 90° C. | 90° C. |
| 1 | Hydroxypropylmethyl Cellulose | Lower than | 88 | No |
| | (Methocel K100M CR) | 90° C. (<90° C.) | | |
| 2 | Hydroxypropyl Cellulose | Lower than | 94 | No |
| | (Klucel HXF) | 90° C. (<90° C.) | | |
| 3 | Polyethylene Oxide | Greater than | 7 | Yes |
| | (Polyox WSR 303-Leo) | 90° C. (>90° C.) | | |

From the above table, it can be concluded that the cloud point of the water soluble ingredient inhibit the extraction of the drug substance form the dosage form.

What is claimed is:

1. A tamper resistant pharmaceutical composition comprising
a. drug substance;
b. water soluble ingredient having cloud point greater than about 90° C.; and
c. optionally at least one pharmaceutically acceptable ingredient;
wherein the cloud point of the water soluble ingredient inhibit extraction of the drug substance from the pharmaceutical composition by hot water having temperature greater than about 90° C.

2. A tamper resistant pharmaceutical composition comprising
a. drug substance;
b. water soluble ingredient, wherein cloud point of the water soluble ingredient is greater than about 90° C.;
c. optionally cloud point modifier in an amount sufficient to increase cloud point of the water soluble ingredient till at least about 100° C.; and
d. optionally at least one pharmaceutically acceptable ingredient;
wherein the drug substance produce an intended therapeutic effectiveness in a subject in need thereof after correct administration of a unit dosage form of the pharmaceutical composition;
wherein the water soluble ingredient inhibit extraction of the drug substance from the pharmaceutical composition by hot water having temperature greater than about 90° C., wherein quantity of the hot water is at least sufficient to dissolve all amount of the drug substance of the unit dosage form.

3. A tamper resistant pharmaceutical composition comprising
a. drug substance;
b. water soluble ingredient having cloud point lower than about 90° C.;
c. cloud point modifier in an amount sufficient to increase cloud point of the water soluble ingredient till at least about 90° C.; and
d. optionally at least one pharmaceutically acceptable ingredient;
wherein the water soluble ingredient and the cloud point modifier inhibit extraction of the drug substance from the pharmaceutical composition by hot water having temperature greater than about 90° C.

4. A tamper resistant pharmaceutical composition of claim 1, claim 2 and claim 3 comprising the water soluble ingredient selected from group consisting of gum, polymer, sugar, salts, dye, irritating agent, salts of organic acid, acid, base, polysaccharide or any combination thereof.

5. A tamper resistant pharmaceutical composition of claim 1 and claim 3, wherein quantity of the hot water is at least sufficient to dissolve all amount of the drug substance of the pharmaceutical composition.

6. A tamper resistant pharmaceutical composition of claim 2 and claim 3, wherein the pharmaceutical composition comprising cloud point modifier selected from group consisting of surfactant, phospholipids, ionic or non-ionic ingredients, glycols, glycol ethers, electrolytes or any combination thereof.

7. A tamper resistant pharmaceutical composition of claim 1, claim 2 and claim 3, wherein the pharmaceutical composition reduce or mitigate potential abuse by making more difficulties in process of extraction of the drug substance from the pharmaceutical composition by hot water having temperature greater than about 90° C. than other pharmaceutical composition comprising a water soluble ingredient having cloud point lower than about 90° C. without cloud point modifier.

8. A tamper resistant pharmaceutical composition of claim 1, claim 2 and claim 3, wherein the pharmaceutical composition further comprising at least one pharmaceutically accepted ingredient selected from group consisting of disintegrant, diluents, binder, lubricant, controlled release ingredient, glidant, surfactant, nasal irritating agent or any combination thereof.

9. A tamper resistant pharmaceutical composition of claim 1, claim 2 and claim 3 is manufacture by non-thermal process or by thermal process, wherein the pharmaceutical composition manufacture by thermal process exhibit higher breaking strength compare to manufacture by non-thermal process.

10. A tamper resistant pharmaceutical composition of claim 9, wherein the pharmaceutical composition manufactured by thermal process exhibit breaking strength at least sufficient to resist pulverisation of the dosage form and thereby not more than 35% pulverized particles of the pharmaceutical composition pass through #200 sieve.
